(19) United States
(12) Patent Application Publication
(10) Pub. No.: US 2023/0390207 A1
(43) Pub. Date: Dec. 7, 2023

(54) INSTANT TRADITIONAL-CHINESE-MEDICINE DRIED POWDER FOR DECOCTION AND METHOD OF PREPARING AND USING SAME

(71) Applicant: Wenmin DU, Calgary (CA)

(72) Inventor: Wenmin DU, Calgary (CA)

(21) Appl. No.: 18/021,720

(22) PCT Filed: Aug. 25, 2020
(86) PCT No.: PCT/CA2020/051159

§ 371 Date: Feb. 16, 2023

(51) Int. Cl. A61K 9/16; A61K 9/00; A61K 36/65; A61K 36/232; A61K 36/236; A61K 36/484; A61K 36/725

(52) U.S. Cl. CPC A61K 9/1694; A61K 9/1664; A61K 9/1652; A61K 9/0095; A61K 36/65; A61K 36/232; A61K 36/236; A61K 36/484; A61K 36/725; A61K 2236/17; A61K 2236/15

(57) ABSTRACT

A method for preparing, from one or more dried traditional-Chinese-medicine materials in non-powder forms, instant traditional-Chinese-medicine dried powder for decoction. The method has the steps of: adding one or more first edible excipients to the one or more dried traditional-Chinese-medicine materials; and grinding the mixture of the one or more first edible excipients and the one or more dried traditional-Chinese-medicine materials to powder with sizes of a predefined range to obtain the instant traditional-Chinese-medicine dried powder. In this method, the one or more first edible excipients are added to the one or more dried traditional-Chinese-medicine materials before or during said grinding. In various embodiments, the edible excipients may be dextrin, glutinous-rice powder, rice powder, and starch. The instant traditional-Chinese-medicine dried powder may be packaged into sealed tea bags or gauze bags for longterm storage up to five years and for convenient taking.

### FIELD OF THE DISCLOSURE

The present disclosure relates generally to a method for preparing and using traditional Chinese medicines, and in particular, to instant traditional-Chinese-medicine dried powder which is ready for decoction, and a method for preparing and using same.

### BACKGROUND

Traditional-Chinese-medicine decoctions or herb-medicine decoctions have been used in China for thousands of years and are widely used today.

Decoction, also known as soup, is one of the earliest and most widely used dosage form in China. An overview of Chinese medicine decoction may be found in the academic paper entitled “The Research Status of Chinese Medicine Decoction and the Major New Types of Chinese Medicine,” by Chen Bin and Zhao Aiguang, published on WORLD CHINESE MEDICINE, March 2014, vol. 9, no. 3.

As decoctions are water-based preparations, they are prone to deterioration or spoilage caused by, e.g., mold or mildew and generally require fresh preparation. The conventional preparation method is to mix the prescribed traditional Chinese medicines into a casserole or a copper pot, decoct the mixture twice each with a suitable amount of water through boiling, combine the two decoctions, separate the combined decoction into two equal doses, and then drink them at separate times. Through decoction, the medical ingredients of the traditional Chinese medicines may be absorbed by human body and takes effect quickly.

Traditionally, there exist standardized prescriptions for treating typical illnesses. Customized prescriptions are also available, in which the traditional-Chinese-medicinal materials may be prescribed based on diagnosis and following the dialectical treatment principle of Chinese medicine (i.e., diagnosis and treatment based on an overall or comprehensive analysis of the illness and the patient's condition). A customized prescription may be modified from a standardized prescription based on diagnosis, in which the traditional Chinese medicines may be altered and/or adjusted by adding and/or removing some traditional-Chinese-medicinal materials, and/or adjusting the quantity thereof, following the dialectical treatment principle of Chinese medicine.

Decoction is often a compound (made of two or more ingredients). Its pharmacological effect is not an aggregation or simple addition of individual pharmacological effects of each traditional-Chinese-medicinal material, but rather a combination thereof. In other words, there exists a compatible function or interaction between the medical ingredients of the traditional Chinese medicines such as “synergism”, “promotion”, and “constraint”, which is known as the art of prescription compatibility of traditional Chinese medicines. For certain prescribed traditional-Chinese-medicinal materials for decoctions, under the condition of heating and boiling, various chemical components undergo complex reactions to produce therapeutic effect different from the aggregation of the original traditional-Chinese—medicinal materials. Therefore, decocting multiple traditional Chinese medicines or herbs together is an intrinsic characteristic of decoction which is a unique feature of Chinese medicine preparation and cannot be replaced by other dosage forms such as pills, powder, slices, paste, “dan” (pellets) and “chong” (granules that can be dissolved in hot water). Generally, it is not preferable to decoct each traditional-Chinese-medicine material separately and mix the decoctions at use.

However, there are specific technical requirements for decocting traditional Chinese medicine and it is sometimes difficult to accurately meet those objective and/or subjective conditions and restrictions. Generally, the decoction's therapeutic effect may be affected the decocting process and other factors such as: the water quality, the amount of time for decocting, the amount of water added, the choice of quick decocting versus slow decocting, and the sequence of putting certain traditional Chinese medicinal materials into water (e.g., some traditional Chinese medicinal materials may need to decoct in cold water before boiling). The decoction is also inconvenient to store and/or carry. Moreover, decoction of traditional Chinese medicine by the users may cost extra time and material.

The traditional decocting method is inconvenient as it difficult to meet emergent needs and difficult to adapt to the modern fast-paced lifestyle. Other dosage forms such as pills, “dan” (pellets), slices, paste, “chong” (granules that can be dissolved in hot water) are only able to solve certain insufficiencies of decoction such as the inconvenience of decocting, and the difficulty to store and carry. However, those dosage forms still have certain limitations and they are usually not as good as the decoction dosage form in terms of the therapeutic effect and in terms of the ability to alter and adjust the prescription based on diagnosis (i.e. to add and/or remove any traditional-Chinese-medicinal material).

With the development of the modern society and the fast pace of modern life, the traditional decocting method has many drawbacks including: lengthy and complicated preparation process, inconvenient to store, carry or transport, and inconvenient to use. Other dosage forms such as the Chinese medicine formula granules has high processing cost, doesn't comply with traditional Chinese medicine principle, and the therapeutic effect may be uncertain or reduced. The dosage form of the Chinese medicine micro-powder has high processing cost and may produce or increase drug toxicity, thus raising medication safety concerns.

Therefore, there is a need for improved decoction dosage form and preparation and use methods thereof, for solving at least some of the many drawbacks associated with the traditional decoction dosage form under the modern conditions and the inability of known dosage forms in the traditional Chinese medicine market to adapt to dialectical treatment (diagnosis and treatment based on an overall analysis of the illness and the patient's condition).

In particular, there is a need to provide an improved traditional-Chinese-medicine decoction dosage form which is:
(a) easy to take and dose without requiring a long time to boil and heat as otherwise required by the traditional method of preparing traditional-Chinese-medicine decoction;
(b) small package, convenient to carry, and convenient to store;
(c) complying with the “Siqi, Wuwei” principle of the Chinese medicine (“Siqi” means four properties: cold as winter, cool as autumn, warm as spring, hot as summer; “Wuwei” means five flavors: sweet, pungent, salty, sour, and bitter), and retaining the property and flavor of the traditional Chinese medicine;
(d) suitable for using the traditional process of decocting (boiling and heating) multiple traditional Chinese medicines together, during which a series of physical chemical reactions will happen to change the pH value of the medicinal liquid, giving rise to an effect of increased solubility, increased efficacy, decreased toxicity, and often improved therapeutic effect;
(e) complying with traditional Chinese medicine's clinical drug usage requirements, keeping its intrinsic characteristic of dialectical treatment features, and allowing the prescription to be altered and adjusted based on diagnosis; as the effectiveness of traditional Chinese medicine is reflected in its clinical dialectical treatments, the prescription may be flexible after careful diagnosis with focus on the patient's overall health condition;
(f) with both the advantages of the traditional Chinese medicine's dialectical treatment and the convenience of taking Chinese patent medicine, complying with the requirement of processing traditional ready-to-use Chinese medicines slices, and keeping their natural flavor; and
(f) simple production process with low cost.

### SUMMARY

According to one aspect of this disclosure, there is provide a method of preparing, from one or more dried traditional-Chinese-medicine materials in non-powder forms, instant traditional-Chinese-medicine dried powder for decoction. The method comprises: adding one or more first edible excipients to the one or more dried traditional-Chinese-medicine materials; and grinding the mixture of the one or more first edible excipients and the one or more dried traditional-Chinese-medicine materials to powder with sizes of a predefined range to obtain the instant traditional-Chinese-medicine dried powder. Said adding the one or more first edible excipients to the one or more dried traditional-Chinese-medicine materials comprises: adding the one or more first edible excipients to the one or more dried traditional-Chinese-medicine materials before or during said grinding.

In some embodiments, the predefined range is 60 to 70 mesh.

In some embodiments, the predefined range is 90 mesh.

In some embodiments, the predefined range is 60 to 100 mesh.

In some embodiments, the one or more first edible excipients are in the form of powder with a size of 100 mesh.

In some embodiments, the one or more first edible excipients comprise at least one of dextrin, glutinous-rice powder, rice powder, and starch.

In some embodiments, said adding the one or more first edible excipients to the one or more dried traditional-Chinese-medicine materials comprises: adding a predefined amount of the one or more first edible excipients to the one or more dried traditional-Chinese-medicine materials before or during said grinding for facilitating grinding and for adjusting dosage.

In some embodiments, said predefined amount of the one or more first edible excipients is such that a dosage of 1 gram (g) of the instant traditional-Chinese-medicine dried powder is equivalent to a dosage of 5 g of the one or more dried traditional-Chinese-medicine materials in the non-powder forms.

In some embodiments, the method further comprises: packaging the instant traditional-Chinese-medicine dried powder in one or more containers with each container receiving a predefined quantity of the instant traditional-Chinese-medicine dried powder.

In some embodiments, the containers are one or more of medicinal bottles, non-woven fabrics disposable tea bags, and gauze bags.

In some embodiments, the method further comprises: sealing the containers; and storing the sealed containers for no more than five years.

In some embodiments, the method further comprises: adding a predefined amount of one or more second edible excipients to the traditional-Chinese-medicine dried powder after said grinding.

In some embodiments, the one or more second edible excipients are in the form of powder with a size of 100 mesh.

In some embodiments, said predefined amount of the one or more second edible excipients is such that a dosage of 1 gram (g) of the instant traditional-Chinese-medicine dried powder is equivalent to a dosage of 2.5 g of the one or more dried traditional-Chinese-medicine materials in the non-powder forms.

According to one aspect of this disclosure, there is provide a method of taking the instant traditional-Chinese-medicine dried powder prepared according to any one of claims 1 to 12. The method comprises:
i) obtaining the instant traditional-Chinese-medicine dried powder;
ii) soaking the instant traditional-Chinese-medicine dried powder in a predefined first amount of water for a predefined first period of soaking time and then heat and boil for a predefined first period of boiling time;
iii) heating and boiling the instant traditional-Chinese-medicine dried powder and the water for a predefined second period of time for obtaining a decoction;
iv) collecting decoction obtained from step iii);
v) adding a predefined second amount of water into a first residue obtained at step iv), and heating and boiling the first residue and the second amount of water for a predefined second period of boiling time;
vi) collecting decoction obtained from step v);
vii) adding a predefined third amount of water into a second residue obtained at step vi), and heating and boiling the second residue and the third amount of water for a predefined third period of boiling time;
viii) collecting decoction obtained from step vii);
ix) combining the collected decoctions and partitioning the combined decoctions into three equal doses;
x) take the three doses in three separate times of a day.

In some embodiments, the first period of soaking time is thirty minutes.

In some embodiments, the first period of boiling time is ten minutes.

In some embodiments, the second period of boiling time is ten minutes.

In some embodiments, the first amount of water is boiling water.

In some embodiments, the three separate times comprise morning, noon, and evening or afternoon.

### BRIEF DESCRIPTION OF THE DRAWINGS

FIG. 1 is a flowchart showing the steps of a method for preparing instant traditional-Chinese-medicine dried powder for decoction, according to one embodiment of this disclosure;

FIG. 2 is a flowchart showing the steps of a method for preparing instant traditional-Chinese-medicine dried powder for decoction, according to another embodiment of this disclosure; and

FIG. 3 is a flowchart showing the steps of a method of making decoction from the instant traditional-Chinese-medicine dried powder and taking the obtained decoction.

### DETAILED DESCRIPTION

Embodiments herein disclose a method for preparing instant traditional-Chinese-medicine dried powder for decoction.

The instant traditional-Chinese-medicine powder may be prepared, administrated, and/or taken in a manner similar to the traditional decoctions and allow the prescription be altered and adjusted based on diagnosis (i.e., adding and/or removing some traditional Chinese medicinal materials, and/or adjusting the quantities thereof). A decoction may be obtained by using the traditional boiling and heating a mixture of the instant powders of multiple traditional Chinese medicines, which keeps the intrinsic characteristic of decoction with a special taking method. Thus, the instant traditional-Chinese-medicine powder disclosed herein provides advantages over known dosage forms of traditional Chinese medicine such as pills, paste and powder.

The instant traditional-Chinese-medicine powder disclosed herein may be packaged in standardized dosage for ease of prescriptions thereby allowing convenience of use. In particular, one or more standardized packages of the instant powders of various traditional Chinese medicines may be prescribed, and may be boiled and heated in water in a regular container for a prescribed time such as 10 minutes. The obtained decoction maintains the useful medicinal ingredient to the greatest extent.

Thus, the instant traditional-Chinese-medicine powder disclosed herein provides a new decoction dosage form that is easy to take, store, and carry, with significant saving of medical materials, and is suitable to adjust the prescription based on diagnosis, thereby better fitting the pace of modern lifestyle with improved therapeutic effect and property than those of the traditional decoction dosage forms. The instant traditional-Chinese-medicine powder disclosed herein allows an effective, convenient and less costly preparation method for a new dosage form in the traditional Chinese medicine market.

Some of the advantages of the embodiments disclosed herein are:
(1) maintaining the characteristics of traditional decoction such as fast and high level of therapeutic effect, suitable for treating a wide variety of illness, and the ability to alter and adjust the prescription based on diagnosis;
(2) overcoming various insufficiencies of traditional decoction such as inconvenience of decocting and the difficulty to store and carry;
(3) suitable for making single-ingredient dried powder for decoction and/or for making any standardized prescription;
(4) suitable for convenient decocting process with a predefined amount of water and shortened and predefined decocting time (e.g., 10 minutes);
(5) ensuring accurate dosage taken each time;
(6) maintaining the intrinsic characteristics of traditional decoction such as the ability to decoct multiple traditional Chinese medicinal materials (i.e. herbs) together and the ability to alter and adjust the prescription based on diagnosis;
(7) allowing the old and traditional decoction dosage form to adapt to the pace of modern lifestyle with improved therapeutic effect.

In some embodiments, the method for preparing the instant traditional-Chinese-medicine powder for decoction comprises grinding the traditional Chinese medicine into powder of predefined sizes. In some other embodiments, the method for preparing the instant traditional-Chinese-medicine powder for decoction comprises mixing suitable excipients with the traditional Chinese medicine and grinding the mixture into powder of predefined sizes. The preparation methods disclosed herein maintains the active ingredient to the greatest degree, simplifies the preparation and decoction processes, reduces the amount of traditional-Chinese-medicine materials used, reduces costs, and is easy to implement.

The methods disclosed herein are suitable for making single ingredient traditional-Chinese-medicine dried powder for decoction and for making a series of standardized, classic or modern prescriptions. The instant traditional-Chinese-medicine powder prepared using the methods disclosed herein may replace the traditional decoction dosage form and/or other dosage forms such as pills, dan (pellet), paste and chong, thereby greatly decreasing the cost for producing Chinese patent medicines.

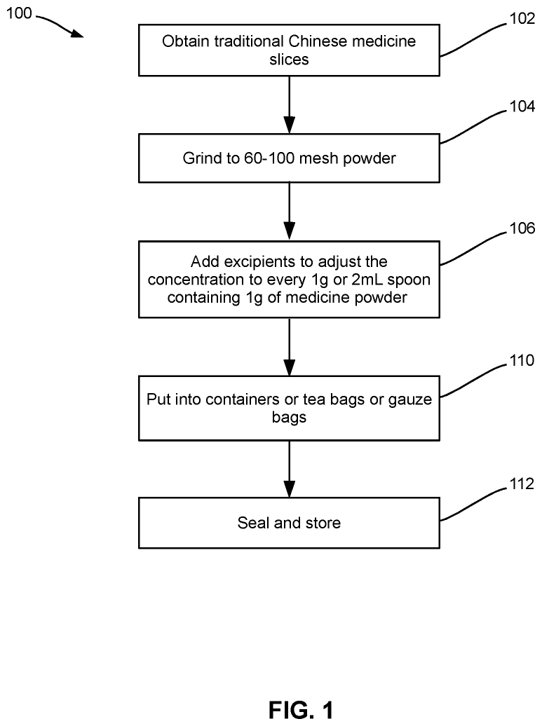

Turning now to FIG. 1, a flowchart showing the steps of a process 100 for preparing instant traditional-Chinese-medicine powder for decoction is shown. The process 100 begins by obtaining a suitable amount of traditional Chinese medicine in a suitable form such as slices (step 102).

Herein, the traditional Chinese medicine may be the full amount of qualified ready-to-use traditional Chinese medicine according to the prescription which indicates what traditional-Chinese-medicine materials are required and the amount required for each material (usually either in grams (g) or in the traditional Chinese measurements of qian (equal to 5 g in modern standard or 3.125 g in ancient standard) and Jiang (equal to 50 g in modern standard or 31.25 g in ancient standard)). As is known in the art, raw or crude traditional-Chinese-medicine materials, mostly herbs, are generally in non-powder forms and are often washed, dried and then sliced into slices with suitable thickness so they are ready-to-use. Such traditional-Chinese-medicine materials may be used at step 102.

In step 104, the traditional-Chinese-medicine materials are then grinded to fine powder using any suitable machines or methods. For example, in one embodiment, all traditional-Chinese-medicine materials are grinded using grinders and blenders and then passing through a 60 to 70 mesh screen or sieve to obtain fine powder with sizes of 60 to 70 mesh (about 250 micrometers (μm) to about 212 μm). In one embodiment, all traditional-Chinese-medicine materials are grinded to fine powder with sizes of about 90 mesh (about 160 μm). In one embodiment, all traditional-Chinese-medicine materials are grinded to fine powder with sizes of 60 to 100 mesh (about 250 micrometers (μm) to about 150 μm).

Those skilled in the art will appreciate that, in some other embodiments, different traditional-Chinese-medicine materials may be grinded to fine powder with different sizes. For example, in one embodiment, one or more first traditional-Chinese-medicine materials may be grinded to fine powder with sizes of 60 to 70 mesh and one or more second traditional-Chinese-medicine materials may be grinded to fine powder with sizes of about 90 mesh.

In step 106, the medicinal powder obtained in step 104 a suitable may optionally be mixed with a suitable amount of excipients in 100-mesh powder form using any suitable machines and methods such as a vibrating mixer for adjusting the concentration of the medicinal powder. The excipients may be selected from edible ingredients such as dextrin, glutinous-rice powder, rice powder, and starch (e.g., cornstarch).

After concentration adjustment, every 1 gram (g) (or measured in a 2 milliliter (mL) spoon) of the concentration-adjusted powder is equivalent to the dosage of 5 g of original traditional-Chinese-medicine slices (the “raw material”).

According to the prescribed amount, the powder obtained in step 106 is then packaged into suitable containers such as bottles, non-woven fabrics disposable tea bags or gauze bags of various sizes (step 110). The final product (medicinal powder) may be stored for up to about five years in sealed conditions (step 112).

In one example, Bai Shao (Radix Paeoniae Alba) may be prepared using the process shown in FIG. 1 to obtain concentration-adjusted Bai Shao powder wherein every 1 g or 2 mL of pure Bai Shao powder is equivalent to the dosage of 5 g of original Bai Shao slices (the “raw material”). The steps are as follows:
Step 102: obtain one pound (454 g) of radix paeoniae alba (raw material).
Step 104: use a Chinese-medicine grinder to grind the obtained radix paeoniae alba for three minutes and pass the powder through a 100 mesh screen to remove coarse particles and obtain about 445 g fine Bai Shao powder.
Step 106: add 450 g glutinous-rice powder and mix for 20 minutes in a vibrating mixer to obtain concentration-adjusted Bai Shao powder.
Step 110: package the concentration-adjusted Bai Shao powder in 200 mL medicinal bottles.

The concentration-adjusted Bai Shao powder has a dosage that every 2 mL spoon (1 g) of the concentration-adjusted Bai Shao powder is equivalent to the dosage of 5 g of original Bai Shao slices.

In another example, Dang Gui (angelica sinensis) may be prepared using the process shown in FIG. 1 to obtain concentration-adjusted Dang Gui powder. The steps are as follows:
Step 102: obtain 1000 g of ready-to-use angelica sinensis slices.
Step 104: grind the angelica sinensis slices to powder and pass the powder through a 100-mesh screen to obtain 970 g Dang Gui powder.

In this example, no excipients are added.

The concentration-adjusted Dang Gui powder has a dosage that every 2 mL spoon (1 g) of the concentration-adjusted Bai Shao powder is equivalent to the dosage of 5 g of original Bai Shao slices. Therefore, a prescription may prescribe the concentration-adjusted Dang Gui powder with a dosage of 20% of that of the Bai Shao slices.

In yet another example, Sheng Hua powder (decoction for postpartum troubles) may be made using the process shown in FIG. 1:
Step 102: obtain 150 g of Dang Gui (angelica sinensis), 90 g of Chuan Xiong (ligusticum wallichii), 90 g of Tao Ren (peach kernel), 15 g of Pao Jiang (prepared or black roasted ginger), and 15 g of Zhi Gan Cao (honey-fried licorice root) (the “raw materials”).
Step 104: grind the “raw materials” to powder and pass the powder through a 100-mesh screen to obtain 350 g of fine medicinal powder.
Step 110: package the fine medicinal powder into 10 bags (e.g., non-woven fabrics disposable tea bags or gauze bags) such that each bag contains 35 g of the fine medicinal powder, and seal the bags for long-term storage (e.g., up to about five years).

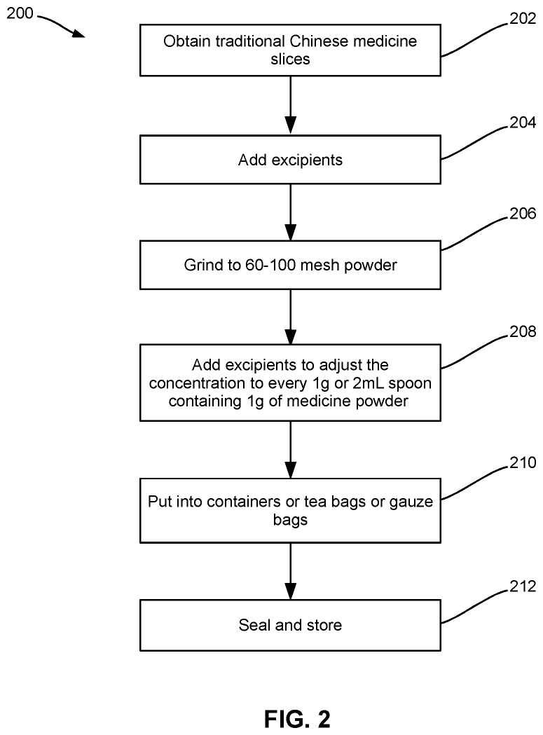

FIG. 2 is a flowchart showing the steps of a process 200 for preparing instant traditional-Chinese-medicine powder for decoction, according to another embodiment. In this embodiment, excipients are added to the traditional-Chinese-medicine materials before grinding for concentration adjustment and improving the grinding performance of the traditional-Chinese-medicine materials, which is useful for grinding “sticky” traditional-Chinese-medicine materials wherein the particles thereof may stick to the grinding component and cause difficulties in grinding the material to powder of desired sizes.

The process 200 is similar to the process 100 shown in FIG. 1. In step 202, traditional-Chinese-medicine slices are obtained and added into the grinder (step 202).

In step 204, a suitable amount of excipients are added into the grinder before or during grinding, for reducing the “stickiness” of the traditional-Chinese-medicine slices and facilitating grinding, and for adjusting the concentration of the medicinal powder. The excipients may be edible ingredients such as dextrin, glutinous-rice powder, rice powder, and/or starch (e.g., cornstarch).

In step 206, the traditional-Chinese-medicine slices and the excipients are grind to to 100 mesh fine medicinal powder. The excipients facilitate the grinding process and allow better extraction of medicinal ingredients through grinding. The amount of excipients added before or during grinding may be recorded.

In step 208, excipients may be added to further adjust the concentration of the medicinal powder to a desired level, for example, every 1 g or 2 mL of the obtained medicinal and excipient powder mixture containing 0.5 g of the medicine powder and equivalent to the dosage of 2.5 g of traditional-Chinese-medicine slices (the “raw material”).

Similar to process 100, the concentration-adjusted powder is then packaged into containers or bags (step 210), and then sealed and stored (step 212).

As those skilled the art will appreciate, in some embodiments, the concentration adjustment may be performed at step 204 by adding excipients for both concentration adjustment and for facilitating grinding. Step 208 is then not required in these embodiments.

In one example, Zao (jujube or date; which is a “sticky” herb) is prepared using the process shown in FIG. 2.
Step 202: obtain one pound (454 g) of jujubes, discard the pip, and cut them to slices of about 5 millimeter (mm) thickness; dry the slices in a drying oven at 70 degree Celsius (158 Fahrenheit) for 10 hours; and obtain the dried jujube slices.
Step 204: add one pound (454 g) of glutinous-rice powder to the dried jujube slices for concentration adjustment and facilitating grinding.
Step 206: put the mixture of dried jujube slices and glutinous-rice powder into a Chinese-medicine grinder and grind for three minutes; pass the mixed medicinal and glutinous-rice powder through a 100-mesh screen to remove coarse particles and obtain 890 g fine medicinal powder.
Step 210: package the final product in 200 mL medicinal bottles.

When prescribing, the dosage of every 1 g or 2 mL spoon of the concentration-adjusted jujube powder is equivalent to the dosage of 2.5 g of original jujube slices.

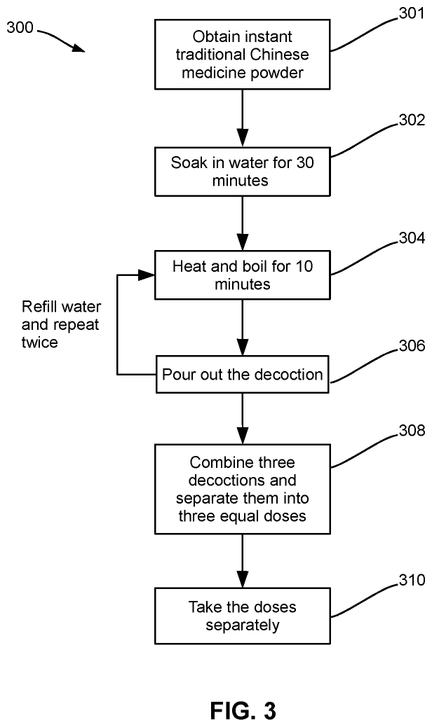

FIG. 3 is a flowchart showing the steps of a process 300 for making and drinking the instant traditional—Chinese-medicine decoction from the above-described concentration-adjusted medicine powder.

Process 300 begins with obtaining a bag of instant traditional-Chinese-medicine dried powder prepared as described above (step 301) and then soak it in water for thirty minutes (step 302). The bag of instant traditional-Chinese-medicine dried powder and the soaking water are heated and boiled for ten minutes (step 304) and the decoction is collected into a separate container (step 306).

Then, steps 304 and 306 are repeated twice with each time after collecting the decoction adding the same amount of water to the residue. In step 308, the three decoctions are combined and the combined decoction is partitioned into three equal doses. In step 310, the patient may take the three doses in three separate times (such as morning, noon and evening) for treatment.

In one example, the Sheng Hua powder (decoction for postpartum troubles) obtained as described above may be used for making the decoction following process shown in FIG. 3: In this example, a patient may take one bag of the instant Sheng Hua dried powder per day.
Step 301: obtain a one bag of the instant Sheng Hua dried powder.
Step 302: put the bag of the instant Sheng Hua dried powder into 400 mL boiling water and soak the instant Sheng Hua dried powder (in the bag) for thirty minutes.
Step 304: heat and boil the water and the bag of the instant Sheng Hua dried powder for ten minutes.
Step 306: collect the decoction into a separate container.

Repeat steps 304 and 306 twice with each time adding another 400 mL of water to the residue; three decoctions are then obtained (each about 400 mL).
Step 308: combine and mix the three decoctions and then partition the combined decoction into three equal doses (each about 400 mL).
Step 310: take the three decoction doses in three separate times of a day (i.e. morning, noon, evening) after shaking and warming.

Although embodiments have been described above with reference to the accompanying drawings, those of skill in the art will appreciate that variations and modifications may be made without departing from the scope thereof as defined by the appended claims.

1-20. (canceled)

21. A method of preparing a traditional-Chinese-medicine product from one or more dried raw traditional-Chinese-medicine materials in non-powder forms, the method comprising:
adding one or more first edible excipients to the one or more dried raw traditional-Chinese-medicine materials; and
grinding the mixture of the one or more first edible excipients and the one or more dried raw traditional-Chinese-medicine materials to powder to obtain the traditional-Chinese-medicine product;
wherein said adding the one or more first edible excipients to the one or more dried raw traditional-Chinese-medicine materials comprises:
adding the one or more first edible excipients to the one or more dried raw traditional-Chinese-medicine materials before or during said grinding.

22. The method of claim 21, wherein said grinding the mixture of the one or more first edible excipients and the one or more dried raw traditional-Chinese-medicine materials to the powder comprises:
grinding the mixture of the one or more first edible excipients and the one or more dried raw traditional-Chinese-medicine materials to the powder with sizes of a predefined range.

23. The method of claim 21, wherein said grinding the mixture of the one or more first edible excipients and the one or more dried raw traditional-Chinese-medicine materials to the powder comprises:
grinding the mixture of the one or more first edible excipients and the one or more dried raw traditional-Chinese-medicine materials to the powder of 60 to 70 mesh, to the powder of 90 mesh, or to the powder of 60 to 100 mesh.

24. The method of claim 21, wherein said one or more first edible excipients are in a form of powder with a size of 100 mesh.

25. The method of claim 21, wherein the one or more first edible excipients comprise at least one of dextrin, glutinous-rice powder, rice powder, and starch.

26. The method of claim 21, wherein said adding the one or more first edible excipients to the one or more dried raw traditional-Chinese-medicine materials before or during said grinding comprises:
adding a predefined amount of the one or more first edible excipients to the one or more dried traditional-Chinese-medicine materials before or during said grinding for facilitating grinding and for adjusting dosage.

27. The method of claim 26, wherein said predefined amount of the one or more first edible excipients is such that a dosage of 1 gram (g) of the traditional-Chinese-medicine product is equivalent to a dosage of 5 g of the one or more dried raw traditional-Chinese-medicine materials in the non-powder forms.

28. The method of claim 21 further comprising:
adding a predefined amount of one or more second edible excipients to the traditional-Chinese-medicine product after said grinding.

29. The method of claim 28, wherein the one or more second edible excipients are in a form of powder with a size of 100 mesh.

30. The method of claim 28, wherein said predefined amount of the one or more second edible excipients is such that a dosage of 1 gram (g) of the traditional-Chinese-medicine product is equivalent to a dosage of 2.5 g of the one or more dried raw traditional-Chinese-medicine materials in the non-powder forms.

31. A traditional-Chinese-medicine product comprising:
traditional-Chinese-medicine dried powder;
wherein the traditional-Chinese-medicine dried powder is prepared from one or more dried raw traditional-Chinese-medicine materials in non-powder forms by:
adding one or more first edible excipients to the one or more dried raw traditional-Chinese-medicine materials; and
grinding the mixture of the one or more first edible excipients and the one or more dried raw traditional-Chinese-medicine materials to powder to obtain the traditional-Chinese-medicine dried powder; and
wherein said adding the one or more first edible excipients to the one or more dried raw traditional-Chinese-medicine materials comprises:
adding the one or more first edible excipients to the one or more dried raw traditional-Chinese-medicine materials before or during said grinding.

32. The traditional-Chinese-medicine product of claim 31, wherein the traditional-Chinese-medicine dried powder has sizes of a predefined range.

33. The traditional-Chinese-medicine product of claim 32, wherein the predefined range is 60 to 70 mesh, 90 mesh, or 60 to 100 mesh.

34. The traditional-Chinese-medicine product of claim 31, wherein the one or more first edible excipients are in a form of powder with a size of 100 mesh.

35. The traditional-Chinese-medicine product of claim 31, wherein the one or more first edible excipients comprise at least one of dextrin, glutinous-rice powder, rice powder, and starch.

36. The traditional-Chinese-medicine product claim 31, wherein the traditional-Chinese-medicine product comprises a predefined amount of the one or more first edible excipients.

37. The traditional-Chinese-medicine product of claim 36, wherein the predefined amount of the one or more first edible excipients is such that a dosage of 1 gram (g) of the traditional-Chinese-medicine dried powder is equivalent to a dosage of 5 g of the one or more dried raw traditional-Chinese-medicine materials in the non-powder forms.

38. The traditional-Chinese-medicine product of claim 31 further comprising:
a predefined amount of one or more second edible excipients.

39. A method of preparing a decoction using the traditional-Chinese-medicine product according to claim 31, the method comprising:
i) obtaining the traditional-Chinese-medicine product;
ii) soaking the traditional-Chinese-medicine product in a predefined first amount of water for a predefined first period of soaking time;
iii) heating and boiling the traditional-Chinese-medicine product and the first amount of water for a predefined first period of boiling time for obtaining a first decoction;
iv) collecting the first decoction obtained from step iii);
v) adding a predefined second amount of water into a first residue obtained at step iv), and heating and boiling the first residue and the second amount of water for a predefined second period of boiling time for obtaining a second decoction;
vi) collecting the second decoction obtained from step v);
vii) adding a predefined third amount of water into a second residue obtained at step vi), and heating and boiling the second residue and the third amount of water for a predefined third period of boiling time for obtaining a third decoction;
viii) collecting the third decoction obtained from step vii); and
ix) combining the collected first, second, and third decoctions and partitioning the combined decoctions into three equal portions.

40. The method of claim 39, wherein the first period of soaking time is thirty minutes;
wherein the first period of boiling time is ten minutes; wherein the second period of boiling time is ten minutes; and/or wherein the first amount of water is boiling water.
